(19) United States
(12) Patent Application Publication
(10) Pub. No.: US 2014/0135601 A1
(43) Pub. Date: May 15, 2014

(54) User replaceable optical subsystem for laser-based photoplethysmography

(71) Applicant: Kestrel Labs, Inc., (US)

(72) Inventors: Jonas Alexander Pologe, Boulder, CO (US); Theodore Philip Delianides, Boulder, CO (US)

(73) Assignee: KESTREL LABS, INC., Boulder, CO (US)

(21) Appl. No.: 13/672,926

(22) Filed: Nov. 9, 2012

(51) Int. Cl. A61B 5/1455; A61B 5/026; A61B 5/145; A61B 5/0295; A61B 5/00; A61B 5/0205; A61B 5/029

(52) U.S. Cl. CPC A61B 5/14552; A61B 5/0205; A61B 5/0261; A61B 5/029; A61B 5/0295; A61B 5/743; A61B 5/14546; A61B 5/02427
USPC 600/324; 29/428

(57) ABSTRACT

A replaceable optical subsystem for a laser-based photoplethysmographic monitor. At least one laser light source (200) is located in an optical subsystem (110) that can be easily detached from the monitor (110). The optical subsystem is attached to the monitor with the aid of a listening element (170) and communicates with the monitor via a monitor connector (160). The patient cable and sensor system, for delivery of the signals to and from the tissue-under-test, attach to the optical subsystem via a sensor connector (150). Other embodiments are described and shown.

### STATEMENT REGARDING FEDERALLY SPONSORED RESEARCH OR DEVELOPMENT

This invention was made with government support under R44 HL073518 awarded by the National Institutes of Health. The government has certain rights in the invention.

### BACKGROUND

1. Prior Art

### U.S. Patents

| Patent Number | Kind Code | Issue Date | Patentee |
|---|---|---|---|
| 5,755,226 | | May 26, 1998 | Carim |
| 5,790,729 | | Aug. 4, 1998 | Pologe |
| 5,891,022 | | Apr, 6, 1999 | Pologe |
| 6,253,097 | B1 | Jun. 26, 2001 | Aronow |
| 6,560,470 | B1 | May 6, 2003 | Pologe |
| 6,647,279 | B2 | Nov. 11, 2003 | Pologe |

2. Background of the Invention

In the science of photoplethysmography, light is used to illuminate or trans-illuminate living tissue for the purpose of providing noninvasive measurements of blood analytes, other hemodynamic parameters, or tissue properties. In this monitoring modality light is directed into living tissue (the so-called “tissue-under-test”) and a portion of the light which is not absorbed by the tissues, or scattered in sonic other direction, is detected a short distance from the point at which the light entered the tissue. The detected pulsatile photoplethysmographic signals are converted into electronic signals that are used to calculate blood analyte levels such as arterial blood oxygen saturation and/or hemodynamic variables such as heart rate, cardiac output, or tissue perfusion. A device which detects and processes photoplethysmographic signals to measure the levels of various blood analytes and/or various hemodynamic parameters is referred to as a photoplethysmographic measurement apparatus, photoplethysmographic device, photoplethysmographic monitor, or photoplethysmographic instrument. The first widespread commercially-used photoplethysmographic device in medicine was the pulse oximeter, a photoplethysmographic device designed to measure arterial blood oxygen saturation.

For over 30 years pulse oximeters have employed light emitting diodes (LEDs), typically housed in the patient sensor, to generate the light used for the measurement of arterial blood oxygen saturation. Unfortunately the light emitted by LEDs can have a full power spectral bandwidth exceeding 60 nanometers (nm), which limits the accuracy and precision with which oxygen saturation can be measured and limits the number of other blood analytes, such as carboxyhemoglobin, that can be accurately measured.

The introduction of laser light sources to photoplethysmography provides the opportunity to expand the field from the measurement of one blood analyte, specifically oxygen saturation, to the measurement of multiple blood analytes and physiological parameters. The narrow spectral bandwidth of laser light improves the spectral resolution, accuracy, and precision of photoplethysmographic measurements, thus making technically feasible the accurate measurement of analytes such as oxyhemoglobin, carboxyhemoglobin, methemoglobin, reduced hemoglobin, and potentially as number of other analytes not yet available through photoplethysmographic measurements. Not unexpectedly, however, the use of lasers in photoplethysmography introduces a number of new problems in the design and implementation of commercially-viable photoplethysmographic instruments. Among these is the fact that it is technically very difficult to position laser light sources in a sensor intended, to be placed directly on the tissue-under-test, particularly when multiple light sources are required. Additionally, any design of a laser-based photoplethysmographic must take into account the possibility that the lasers may have a short enough lifetime that they may need to be replaced before the end of life of the photoplethysmographic monitor.

The difficulty in positioning the laser light sources at the sensor in a photoplethysmographic instrument is due to a combination of elements. The physical size of the laser devices and their mounts may be too large for placement in a conventional finger sensor. The lasers typically must be placed in direct contact with heat spreading and heat sinking mechanical components. Controlling the laser temperature in a photoplethysmographic device can increase measurement accuracy but adds to the need to position the lasers in close proximity to certain thermal control components such as a thermoelectric cooler. Furthermore, given the cost of semiconductor lasers, by comparison to LEDs, it is advantageous to keep the lasers out of the sensor or cable because it reduces operating costs of the instrument by protecting the lasers from damage due to the physical abuse typically experienced by sensors and cables in a clinical setting. Even if it were possible to position the lasers and their respective circuitry and hardware in the sensor or cable, replacing all of these components each time they were damaged or worn out could greatly increase the expense associated with operating such an instrument.

If the laser light sources are not housed at the sensor, the light emitted by the laser (or lasers) must be transmitted from the laser housing to the tissue-under-test. This is typically accomplished by employing one or more light guides. The light guide may be any one of a number of elements, or a chain of elements, including optical elements such as glass or plastic optical fibers, liquid-filled tubes, fiber optic bundles, or other light pipes. The photoplethysmographic signal returning from the tissue-under-test can be in the form of optical signals, i.e. returning via another light guide, or as an electronic signal generated by a photodetector located on the tissue. Such a system requires cabling and connectors for both electrical and optical signals. These two types of signals can also be transmitted in a combined manner via a series of hybrid electrical and optical cables and connectors.

The use of lasers in photoplethysmography was originally proposed nearly two decades ago; however, no laser-based photoplethysmographic monitors have yet been made commercially available. One of the reasons for the delay in the commercial. introduction of laser-based photoplethysmography is the unique challenge of how to properly implement lasers in these types of devices. In a multi-analyte monitor, multiple laser light sources must be used. The electronic and mechanical packaging of these emitters should be simple and low cost, with appropriate physical and electrical protection for both the lasers and associated electronics and emitter-coupled light guides. Additionally, once a photoplethysmographic device is placed into service, it would be convenient to be able to introduce new emitters centered at different wavelengths, thereby allowing the measurement of additional analytes. It would also be convenient to be able to replace a damaged laser without returning the entire photoplethysmographic device for servicing by the manufacturer.

The Minolta/Marquest Model SM-32 Oxygen Saturation Monitor was perhaps the first pulse oximeter put into clinical use, predating even conventional “LED-based” pulse oximeters. Unlike current LED-based photoplethysmographic devices, where light sources (typically LEDs) are located in the sensor and held adjacent to the tissue-under-test, the Minolta/Marquest monitor used a broadband tungsten light bulb, a series of optical filters, and two fiber optic bundles to deliver light to, and receive light from, the tissue-under-test. The finger sensor that held the light guides against the tissue-under-test was detachable from one or both of the fiber optic cables, i.e. either the emitter cable and/or the detector cable, for ease of replacement of the sensor or the light guides should they become damaged or worn. Because of the limited lifetime of filament-based light bulbs, an access door was designed into the monitor to allow the end user to easily replace the light bulb when it burned out. Replacing a light bulb, or designing an instrument such that the light bulb could be easily replaced by the end user, was fairly easy to accomplish because a light bulb is a single discrete element that is always built into a free standing housing, or bulb.

Light bulbs are well known to have limited lifetimes and have, since conception, been implemented as user replaceable devices. In contrast, lasers have typically not been designed to be user replaceable. Whether installed in a compact disc (CD) player, a digital video disc (DVD) player, a laser pointer, or a scientific instrument such as an FT-IR spectrophotometer, the laser portion of these devices is not user replaceable. The entire device must either be sent for service or discarded and replaced with a new one.

U.S. Pat. Nos. 5,790,729, 5,891,022, and 6,560,470 all address various aspects of laser-based photoplethysmographic instrument design and show the laser light sources located in a housing or optical module within the main monitor electronics box. None of these patents present or discuss a design whereby the laser section can be quickly and easily replaced, particularly by an end user in the field, without the need to disassemble and then rebuild, the photoplethysmographic instrument.

U.S. Pat. No. 6,253,097 is a laser-based photoplethysmographic device using Vertical Cavity Surface Emitting Laser (VCSEL) light sources. The abstract of the patent states “The VCSELs are located either in: (1) the probe itself, (2) the connector to the probe, or (3) the monitor box connected with an optical fiber to the probe.” The first two arrangements would require disposing of the entire cable or sensor, i.e. including the expensive the VCSEL emitters, when these items wear out or become damaged. No details are provided on how the VCSELs would be installed if they were located in or on the monitor enclosure, nor does the patent present a design whereby the laser section could be readily replaced by an end user in the field.

U.S. Pat. No. 6,647,279 discusses emitters that might be located either in the main monitor or in the cable or sensor. The Specification states “In the preferred embodiment, the emitters housed in the instrument are contained in the Laser Module 14. This module contains a set of laser diodes that are coupled into a fiber, a fiber bundle, or some other type of light guide 16, for transmission to the sensor and on to the tissue-under-test.” The patent does not discuss whether this laser module could be a removable part of the monitor nor does it present a design whereby the laser section could be easily replaced by an end user in the field.

Locating the laser emitters of a laser-based photoplethysmographic instrument in the cable or sensor subjects them to vibration and other mechanical abuse and introduces additional complexity and cost to the items most likely to wear out from normal clinical use. Placing multiple laser emitters in either the sensor or patient cable also requires multiple electrical conductors in the cable for driving, the emitters and controlling any required temperature stabilization electronics, thereby increasing the cost, size, and complexity of the connector and cable between the monitor and the lasers. Furthermore, a design where the optical emitter module detaches from a section of the cable requires the use of two additional connectors in the system between the monitor and the sensor, further increasing the expense of such a system.

U.S. Pat. No. 5,755,226 is a photoplethysmographic-based system for measuring hematocrit in the blood. This patent shows only a proposed functional arrangement for the device. The specification states that “FIG. 2 shows a simplified block diagram of the present system 100 for noninvasive determination of hematocrit. System 100 includes an optics module 200, an electronics module 400 and a processing module 500,” The block diagram of the optics module in this patent does not show any details of how it would be constructed or how it would integrate into the main instrument. Furthermore, this patent does not disclose the optics module as being a user replaceable component.

Whereas conventional pulse oximetry using LED-based sensors has been in use for many decades, the field of laser-based photoplethysmography is still in its nascent stage. The field holds great promise for the ability to measure multiple blood analytes from a single sensor site, but there are various technology implementation challenges that must be overcome before successful products will reach the market. Foremost among these is the integration of the laser light sources into the instrument. A photoplethysmographic device using laser-based emitters requires the addition of numerous components, including: circuitry for control and stabilization of the laser drive currents; circuitry for protection from electrostatic discharge (ESD), damage to the lasers and associated electronics; mechanical mounts for the laser semiconductor chips designed to rapidly conduct and spread heat generated by the semiconductor junctions; thermal control devices and heat sinks; fibers or other light guides for transmitting light from the lasers; and packaging to mechanically protect the components. For electrical and mechanical reasons that are obvious to one skilled in the art, many of these components must be included with the lasers if the lasers are to be built into a user-replaceable optical subsystem or optical module.

### BRIEF SUMMARY OF THE INVENTION

In accordance with one embodiment a user replaceable optical subsystem for a photoplethysmographic device comprises one or more light sources, including at least one laser, and additional connector features, for interfacing to the main instrument and to the sensor, or sensor cable, arranged in an optical subsystem with included features that allow the detachment of the subsystem and its included light sources from the main instrument. Accordingly, several advantages of one or more aspects are as follows: that the optical subsystem and its included laser light sources can be quickly and easily replaced by simply detaching an old subsystem and attaching a new one, without the need to disassemble and rebuild the entire photoplethysmographic device; that the included connector features provide all electrical and optical interconnections required for operating the optical subsystem as part of the photoplethysmographic instrument; that the laser-based photoplethysmographic device is designed to minimize operating costs while maximizing measurement accuracy and upgradability; and that the inclusion of a replaceable optical subsystem permits the expeditious introduction of new laser sources with different center wavelengths to provide improved accuracy or new measurement capabilities. The combination of these advantages contributes to the creation of a useful device for accurate, high-resolution photoplethysmographic measurements. These and other advantages of one or more aspects will become apparent from review of the following description and the accompanying drawings.

### DRAWINGS

FIG. 1. Photoplethysmographic Device with User Replaceable Optical Subsystem

FIG. 2. User Replaceable Optical Subsystem

FIG. 3. Fastening Elements for a User Replaceable Optical Subsystem

FIG. 4A. Photoplethysmographic Device with Internal User Replaceable Optical Subsystem

FIG. 4B. Internal User Replaceable Optical Subsystem

### DETAILED DESCRIPTION OF THE INVENTION

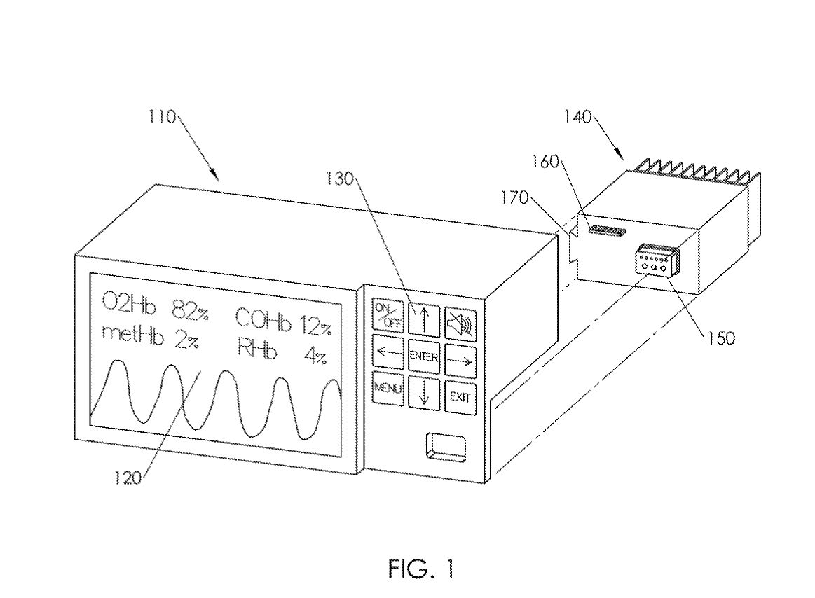

One embodiment of a user replaceable optical subsystem for a laser-based photoplethysmographic device is shown in FIG. 1. A monitor 110 includes a visual display 120 which presents the monitored blood analytes, physiological parameters, waveforms, and other information to the clinician or end user of the monitor. Monitor 110 also includes a user control panel 130 for controlling the operation of monitor 110. An optical subsystem 140 that contains at least one laser light source is mounts, or removably connects, to the monitor 110 with the aid of a fastening element 170 and connects to the monitor via a monitor connector 160. The removably attachable optical subsystem 140 includes a sensor connector 150 that is used to connect a cable and sensor assembly (not shown) for delivering light to, and receiving signals from, the tissue-under-test.

The apparatus of FIG. 1 provides a quick way for replacing the light sources, or emitters, in a laser-based photoplethysmographic device such as a pulse oximeter or other patient monitor. The monitor 110 is the primary electronics box for the device and contains user interface elements and electronics, which could include power supplies, central processing circuitry, and software, while, in this embodiment, the detachable optical subsystem 140 is the light-generating optics module for the system. This optical subsystem would typically house optical components including emitters, such as light-emitting diodes (LEDs), filament lamps, or lasers; light coupling optics; and light guides to aid in transmitting light to the tissue-under-test. The optical subsystem could also include various mechanical and electrical components to aid its functionality. Note that a wide variety of different types of lasers could potentially be used, but most commonly the lasers types would be devices such as semiconductor or diode lasers or vertical cavity surface emitting lasers (VCSELs).

Photoplethysmographic devices such as pulse oximeters are seen in clinical use as both freestanding, or stand alone, devices or as subsystems that are part of larger multi-function, or integrated, patient monitors. These subsystem photoplethysmographic devices may themselves be modules or printed circuit assembly boards that are detachable from the multi-function patient monitor. If the photoplethysmographic device is a subsystem of a larger monitor, the photoplethysmographic device may also share a common user interface, or control panel and display interface, or visual display, with other subsystems that perform patient measurements of additional physiological parameters. Note that the removably attachable optical subsystem 140 would be installed in this multi-function patient monitor in a similar manner to that described herein for its installation in the stand alone monitor 110 shown in FIG. 1.

In the embodiment shown in FIG. 1, the interconnection provided by monitor connector 160 located between the monitor 110 and the optical subsystem 140 would allow for a photoplethysmographic device that is ready to accept a sensor cable or cable/sensor assembly, attached via sensor connector 150, and ready for use for physiological measurements on a patient. The monitor connector 160 could be part of a mating connector pair, or alternatively a length of interconnection cabling or wires with connector terminations or multiple discrete interconnection wires that provide the required interconnections. In the preferred embodiment shown in FIG. 1 the monitor connector 160 is a multi-pin, card-edge connector.

In the embodiment shown in FIG. 1, sensor connector 150 is the primary point of connection for the sensor cable which, operation, would consist of a patient sensor and any associated patient cable. This sensor and patient cable could be two or more discrete elements, or the sensor could be permanently attached to the patient cable, or the sensor could be a sensor without a cable but including an integrated connector for mating directly with sensor connector 150.

The use of at least one fastening element 170 allows the optical subsystem 140 to be easily and reversibly, or removably, attached to the monitor 110. Various fastening elements, or a combination of elements, be employed, including rails, grooves, bosses, dovetails, cradles, discrete fasteners, or latches. The fastening can be achieved either by tightening hold-down screws or from friction designed into close-tolerance sliding or latching parts. This demountable attachment allows easy field replacement or detachment of the optical subsystem, even by an untrained, non-technical end user, including nursing or other clinical personnel, without requiring a complete overhaul or rebuilding of the photoplethysmographic monitor. Replacement of the optical subsystem might be desirable for such purposes as to replace a failed laser or to install a new or different set of laser wavelengths.

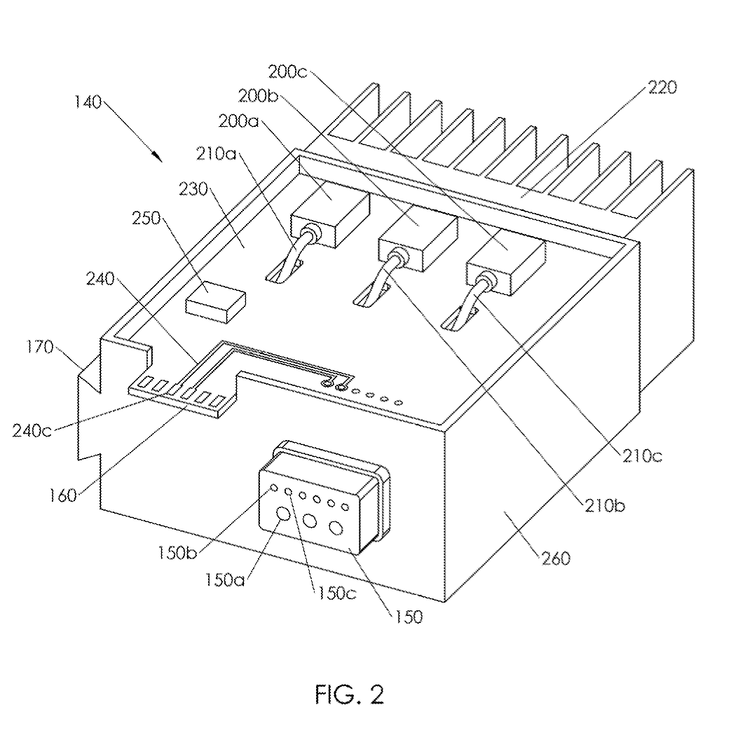

FIG. 2 is a more detailed view of the optical subsystem 140 with its sensor connector 150, monitor connector 160, and fastening element 170. In this embodiment a number of emitters 200a, 200b, and 200c, at least one of which is a laser, are mounted on a printed circuit board assembly 230 located within the optical system. The emitters 200a, 200b, and 200c are coupled to light guides 210a, 210b, and 210c, and these light guides might be optical fibers, liquid-filled tubes, or other types of light pipes. While the embodiment of FIG. 2 shows three emitters, obviously the exact number of emitters required for a specific photoplethysmographic device is dependent on the number of analytes to be measured and the optical extinction of those analytes.

In an alternate embodiment, one or more emitters would be mounted inside an emitter housing (not shown here), internal to the optical subsystem. The emitter housing could also contain various mounts, heat spreaders, coupling optics, and hermetic or quasi-hermetic packaging elements. This emitter housing located on, or internal to, the optical subsystem would thus be replaced any time that the removably attached, or removably connected, optical subsystem is replaced.

The emitters 200a, 200b, and 200c along with other light sources within the optical subsystem, might require circuitry for controlling drive currents or maintaining the device case temperature, and all or part of this circuitry could be co-located on the printed circuit board assembly 230. Device temperature control might be aided by thermoelectric control elements such as a thermoelectric cooler (TEC) (not shown) and its associated heat sink 220.

The printed circuit board assembly 230 includes various electrical elements and design features used in the operation of the optical subsystem. The optical subsystem is in electrical communication with the main photoplethysmographic electronics box 110 via the monitor connector 160. This monitor connector would typically have multiple contacts for transferring signals both to and from the optical subsystem and the patient cable and sensor, when a sensor is attached to the optical subsystem via the sensor connector 150. Design elements in monitor connector 160 or sensor connector 150 or nearby circuitry of printed circuit board 230 could provide electrostatic discharge (ESD) protection for the lasers or other electronic circuitry located within the optical subsystem.

The laser-based photoplethysmographic system of this embodiment employs light guides for delivery of the laser light to the tissue-under-test. The returning photoplethysmographic signals could be either electrical or optical, and there might be additional electrical conductors within the cable and sensor system for photodetector signals, sensor identification circuitry, or drive current lines for additional emitters located on the sensor, thus the cable used in this system would have both optical and electrical conductors. Such a cable would be a hybrid electrical and optical cable; and, similarly, the sensor connector 150 shown in this embodiment is a hybrid electrical optical, or electro-optical, connector. The hybrid electrical optical sensor connector 150 includes at least one light guide, or optical conductor, 150a and at least one electrical conductor 150b. The light guide 150a could connect to the light guide 210a coupled the laser 200a, and the electrical conductor 150b could attach to other electronics within the optical subsystem, for example a component or electrical trace on the printed circuit board assembly 230.

it would also be possible to configure at least one signal path or trace 240 to pass directly through the optical subsystem from its connection 240c at the monitor connector 160 to electrical conductor 150c at the sensor connector 150. This direct signal path could be either an electrical conductor or an optical light guide path; but, by being a direct connection, the signal it would carry, whether electrical, or optical, could pass through the optical subsystem without being altered. There might be electrical resistance or optical attenuation losses, but the signal would not be amplified, filtered, or otherwise significantly altered by passing through the optical subsystem. An example of this type of unaltered signal path connection would be the passage of electrical signals from the photodetector located in the patient sensor through the patient cable and into sensor connector 150, through the optical subsystem, and out the monitor connector 160 until finally reaching the electronics in the monitor 110, where the signal is processed for performing the required photoplethysmographic measurements.

An additional element that can be added to the optical subsystem is a memory element 250. This memory element, which might be an electrically erasable programmable read-only memory (EEPROM) or similar device, can hold important information about the optical subsystem and communicate this information to, or receive new information from, the main photoplethysmographic monitor 110 when the optical subsystem 140 is installed. This information could include emitter center wavelengths, emitter drive currents, temperature setpoints, usage time counters, or any other information that is useful for the proper operation of the photoplethysmographic device. If an entirely new set of laser wavelengths is installed in an optical subsystem, the memory element could provide the monitor with information required so that the correct physiological parameters can be accurately calculated and displayed.

Because the optical subsystem 140 includes emitters, including at least one laser, light guides, and related electronics, it would be advantageous to protect the optical subsystem from damage, either from normal device use or when handling the optical subsystem whenever it is replaced. In the embodiment shown in FIG. 2, the optical subsystem is protected from damage by enclosing it in a housing 260. This could be either an injection molded plastic, a metal box, or any similar element that covers all or part of the optical subsystem. For example, the entire optical subsystem can be enclosed in a box or alternatively a cover can be mounted on the subsystem, with openings as required for the connectors and other items or for providing ventilation, but which still sufficiently shields the optical light guides from breakage or guards the electronic circuitry from physical or ESD damage. Another arrangement would be to use a housing 260 to provide the required barrier to damage for the optics and electronics but allow a heat sink 220 that is part of the temperature control system for the emitters to be exposed to the ambient air to facilitate heat exchange.

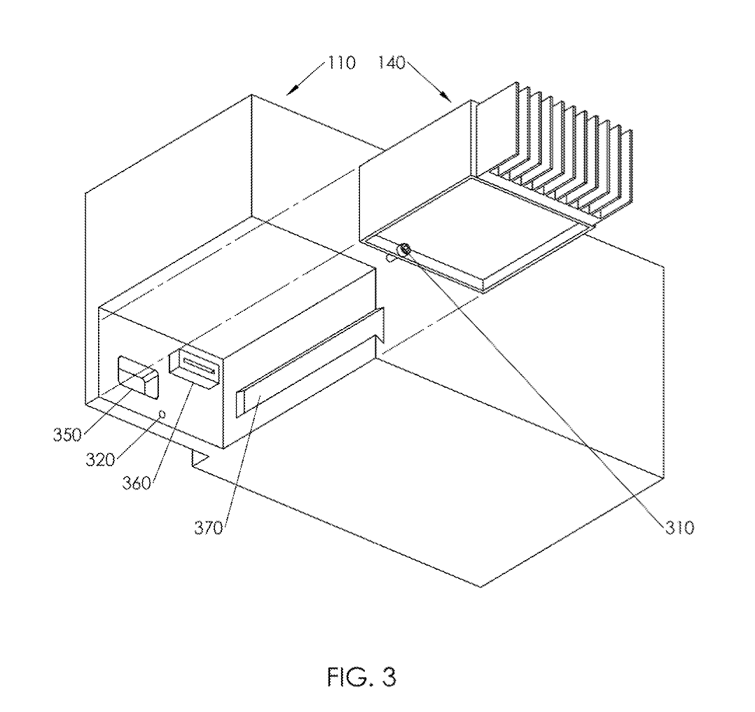

FIG. 3 shows a rear view of monitor 110 and optical subsystem 140, revealing the details of one embodiment of a combination of fastening elements of a user replaceable optical subsystem. The fastening element 170 (previously shown in FIGS. 1 and 2) of the optical subsystem 140 engages with a receiving element 370 located on the monitor 110. The monitor connector 160 of the optical subsystem engages with a receiving connector 360 located on the monitor. In the embodiment shown, the sensor connector 150 protrudes through monitor opening 350 to be accessible to the end user at the front of the monitor. Practically, however, sensor connector 150 could be located on the front of the monitor, on any side, or in a number of other orientations. In the embodiment shown in FIG. 3, a second fastening element is employed in the form of a discrete fastener 310 located on the optical subsystem that mates to a fastener hole 320 in the monitor. In this embodiment, the fastening element 310 screws into the fastener hole 320 to lock the optical subsystem 140 into monitor 110. This fastening element could alternatively be a screw, clip, cross pin, cotter pin, or other type of fastener, and the locations of the discrete fastener and its mating hole could be reversed on the optical subsystem and the monitor. This would, for example, allow the use of a captive screw in the optical subsystem and a threaded hole, fastener hole 320, to be used as a fastener receiving element in the monitor. The discrete fastener could be selected such that it can be operated using a common tool such as a flat-head or Phillips screwdriver or a hexagonal driver. Allowing the fastener to be actuated with a simple tool, which could also include operation by a coin or the fingertips, would facilitate easy removal and replacement of the optical subsystem by untrained clinical personnel who may not have access to specialized tools or equipment.

An additional advantage would be gained by orienting the mating direction of the monitor connector of the optical subsystem with any fastening elements used to hold the optical subsystem securely to the monitor. For example, by aligning the slide direction of fastening element 170 which mates with receiving element 370 (shown for example as a dovetail groove in FIG. 3), and the drive axis of discrete fastener 310, which mates with fastener hole 320, to the insertion direction of monitor connector 160, which mates with receiving connector 360, all three pairs will engage concurrently when the optical subsystem is installed. This co-aligned orientation further simplifies the installation or removal of the optical subsystem by untrained personnel.

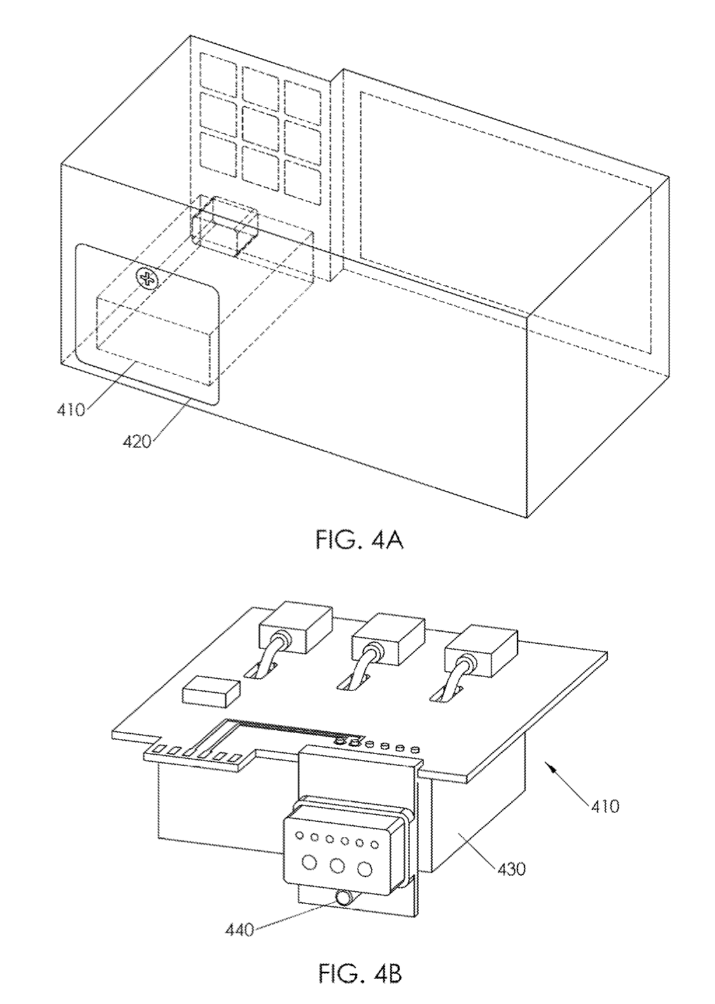

One of many possible alternate embodiments is shown in FIG. 4A, where the optical subsystem is designed to be installed inside the monitor, but can still be easily removed by including the aforementioned monitor connector, sensor connector, and a fastening element as part of internal optical subsystem 410. In this embodiment the internal optical subsystem 410 is located behind an access panel 420. The access panel could be a sliding panel, hinged door, or separate removable part and might be held in place by a discrete fastener, a built-in latch, or a combination thereof. Additional details of an internal optical subsystem are shown in FIG. 48. Because the internal optical subsystem 410 would be protected in part by the main monitor enclosure, a partial housing 430 can be included, for example to protect the light guides. Fastening elements such as discussed previously could be used for removably attaching, or removably connecting, the internal optical subsystem in place, including, for example, an internal fastening element 440. The apparatus could alternatively be designed such that the access panel 420 shown in FIG. 4A provides the fastening element or elements used to securely hold the internal optical subsystem m place.

The previous discussion of the embodiments has been presented for the purposes of illustration and description. The description is not intended to limit the invention to the form disclosed herein. Variations and modifications commensurate with the above are considered to be within the scope of the present invention. The embodiments described herein are further intended to explain the best modes presently known of practicing the invention and to enable others skilled in the art to utilize the invention as such, or in other embodiments, and with the particular modifications required by their particular application or uses of the invention. It is intended that the appended claims be construed to include alternative embodiments to the extent permitted by the prior art.

1. A photoplethysmographic device including an optical subsystem the optical subsystem, the optical subsystem comprising:
a. one or more light sources;
b. a sensor connector for connecting a sensor cable to the optical subsystem;
c. a monitor connector for connecting the optical subsystem to a monitor;
d. at least one of the light sources consisting of a laser; and
e. at least one fastening, element for removably attaching the optical subsystem to the monitor.

2. The device of claim 1 wherein the sensor connector includes one or more electrical conductors and one or more light guides.

3. The device of claim 1 further including at least one signal path configured to pass a signal substantially unaltered through the optical subsystem.

4. The device of claim 1 wherein the at least one fastening element is operable by a simple tool.

5. The device of claim 1 further including a memory element.

6. The device of claim 1 further including a housing for substantially enclosing the optical subsystem.

7. The device of claim 1 wherein the monitor connector is positioned within the optical subsystem to engage concurrently with installation of the optical subsystem to the monitor.

8. A method of manufacturing an optical subsystem of a photoplethysmographic device, the method of manufacturing the optical subsystem comprising the steps of:
a. providing one or more light sources;
b. providing a sensor connector for connecting a sensor cable to the optical subsystem;
c. providing a monitor connector for connecting the optical subsystem to a monitor;
d. selecting a laser for at least one of the one or more light sources; and
e. providing at least one fastening element for removably connecting the optical subsystem to the monitor.

9. The method of claim 8 further comprising the step of including one or more electrical conductors and one or more light guides in the sensor connector.

10. The method of claim 8 further comprising the step of providing at least one signal path for passing a signal substantially unaltered through the optical subsystem between the sensor connector and the monitor connector.

11. The method of claim 8 further comprising the step of designing the at least one fastening element to be operable by a simple tool.

12. The method of claim 8 further comprising the step of providing a memory element to provide information on the optical subsystem to the monitor.

13. The method of claim 8 further comprising the step of substantially enclosing the optical subsystem in a housing.

14. The method of claim 8 further comprising the step of positioning the monitor connector within the optical subsystem to engage concurrently with installation of the optical subsystem to the monitor.

15. A photoplethysmographic device including an optical subsystem, the optical subsystem comprising:
a. one or more laser light sources;
b. a hybrid electrical optical cable connector for connecting a sensor cable to the optical subsystem;
c. an interface connector for connecting the optical subsystem to a monitor;
d. at least one fastening element for removably attaching the optical subsystem to the monitor; and
e. a protective housing that substantially encloses the optical subsystem.
